(19) United States
(12) Patent Application Publication
(10) Pub. No.: US 2007/0134594 A1
(43) Pub. Date: Jun. 14, 2007

(54) Fluorescent dye and structure and manufacturing method of fluorescent storage media using thereof

(76) Inventors: Ming-Chia Lee, Ta-Chia (TW); Wen-Yih Liao, Taichung City (TW); Huei-Wen Yang, Chung-Li (TW); Ching-Yu Hsieh, Chiunglin Shiang (TW); Chien-Liang Huang, Taoyuan (TW); Tzuan-Ren Jeng, Mei-Chih-Cheng (TW); Andrew Teh Hu, Hsinchu (TW); Chien-Wen Chen, Shinyuan Shiang (TW); Chung-Chun Lee, Lun-Pei Hsiang (TW)

Correspondence Address: J.C. Patents, Suite 250, 4 Venture, Irvine, CA, 92618, US

(21) Appl. No.: 11/410,750

(22) Filed: Apr. 24, 2006

Related U.S. Application Data
(62) Division of application No. 10/411,055, filed on Apr. 9, 2003, now abandoned.

(30) Foreign Application Priority Data
Dec. 31, 2002 (TW) 91137969

(51) Int. Cl. G11B 7/24; C07D 213/00

(52) U.S. Cl. 430/270.18; 430/945; 427/164; 427/162; 546/329

(57) ABSTRACT

A fluorescent dye, a structure of a fluorescent storage media and method using thereof, are disclosed. The fluorescent dye of the present invention comprises an organic violet fluorescent compound having a chemical structure (I) is suitable for using a short wavelength laser having a wavelength less than 500 nm as an excitation source. When a short wavelength laser is used for exciting the organic violet fluorescent compound (I), a fluorescence having an emission wavelength larger than 500 nm is induced, and a reading signal can be provided by detecting the intensity of the fluorescence radiation.

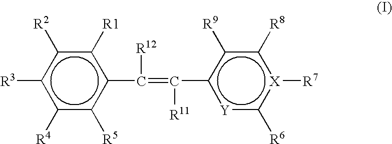

### CROSS-REFERENCE TO RELATED APPLICATION

This application claims the priority benefit of Taiwan application serial no. 91137969, filed on Dec. 31, 2002.

### BACKGROUND OF THE INVENTION

1. Field of the Invention

The present invention generally relates to a fluorescent storage media. More particularly, the present invention relates to a fluorescent dye, a structure and manufacturing method of fluorescent storage media using thereof.

2. Description of the Related Art

With the advent of the generation of information and multimedia, the storage density and capacity requirements of the storage media of the consuming electric products of 3C (Computer, Communication, Consumer Electronics) are growing ever since. In this respect, the information storage media generally uses a red laser as a reading source, because the laser source has a limitation due to optical diffraction, and therefore the storage density is limited. At present some principle and method of enhancement of the storage density of the optical information storage media has been set forth. Such principles and methods include shifting of the wavelength of the reading laser source, such as the shifting of the laser source from red laser to blue laser, or enhancement of the numerical aperture (“NA”) of optical lens. Some other technologies include improvement of the encoding methods of the digital signal, or a disc storage method using an extra-fine resolution near field optical structure, or a technology of increasing the storage capacity of the information storage media (e.g., a compact disc) by using stacked multiple recording layers for forming a storage media, i.e., the recording layers of the information storage media is developed into a three dimensional space multilayer structure, to increase the storage capacity. All the methods described above may increase the storage density effectively.

In an aspect of the shortening of the wavelength of the laser source, a new generation of a high density disc storage specification (Blue-ray Disc) is published in 2002 by companies of Hitachi, LG, National, Pioneer, Philips, Samsung, Sharp, Sony and Thomson Multimedia in common. A single-side Blue-ray Disc may be promoted up to 27 GB by using a 405 nm blue laser source and a 0.1 mm optical transmission cover layer structure. Thus, it can be seen that using a short wavelength laser for reading and saving operations will become the main stream of the development of the storage capacity of the information storage media.

Furthermore, the number of layers of the multiple layer structure of a conventional storage media technology of three dimensional space multilayer structure is limited due to a common frequency destructive interference effect. In 1989, D. A. Pathenopoulos has set forth first that using a detection of the laser excited fluorescence emission strength of a organic discolor material under a variety of states, the common frequency destructive interference problem of the multiple layer structure in a disc has been overcome. Afterwards, Russell further developed a multilayer fluorescence optical storage media and the reading light path system (U.S. Pat. No. 5,278,816). And, in 2001 the Constellation 3D company of the United State announced a fluorescent multilayer disc (“FMD”) having a capacity of 140 GB with a fluorescence absorption of a 650 nm wavelength red laser and emission of a 680 nm wavelength fluorescence. The fluorescent multilayer disc (FMD) uses a fluorescent dye for the dye of the multilayer of the disc.

When the laser source irradiates on the fluorescent dye, a fluorescence having a different wavelength with the laser beam is released, the detector of the disc driver will search the fluorescence and ignore the laser beam. Because the fluorescence does not cause the problem of the common frequency destructive interference, an increased number of recording layers in a multiple layer structure may be used in a single side of a disc. The technology described above shows that, if a short wavelength laser for reading and saving operations and a fluorescence multilayer storage media are combined, the unit area of the storage capacity of a disc may be promoted to a further step. Therefore, a proper fluorescent dye for a short wavelength laser is an important development field for further enhancement of storage capacity for making a high density storage media.

### SUMMARY OF THE INVENTION

Accordingly, the purpose of the present invention is to provide a fluorescent dye, a structure of a fluorescent storage media and a method using thereof, which storage media has a capability to use a short wavelength laser having a wavelength less than 500 nm for the recording the information and replaying the recorded information.

It is another object of the present invention to provide a fluorescent dye, a structure of a fluorescent storage media and a manufacturing method using thereof, which has a capability to use a short wavelength laser having a wavelength less than 500 nm for an excitation source of a fluorescent storage media to enhance the storage capacity of a fluorescent storage media.

It is another object of the present invention to provide a fluorescent dye, a structure of a fluorescent storage media and a method using thereof, which has a capability of substantially reducing the cross-talk between the excitation source and the fluorescence signal and the attenuation of the fluorescence signal due to the increase in the number of recording layers in a storage media disc.

In order to achieve the above objects and other advantages of the present invention, a fluorescent dye, a structure of a fluorescent storage media and a method using thereof is provided. The fluorescent dye of the present invention includes a compound having a following chemical structure (I):

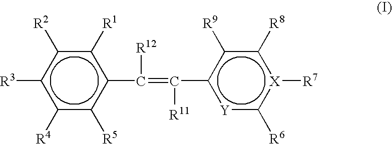

wherein X may be a carbon atom or a nitrogen atom. Y may be a carbon atom attaching a side chain (i.e. C—R⁹) or an oxygen atom. R¹, R², R³, R⁴, R⁵, R⁶, R⁷, R⁸ and R⁹ represent same or different chemical groups, and may be one selected from a group comprising hydrogen atom, halogen atom, substituent alkyl groups with carbon number one to eight (C₁₋₈), non-substituent alkyl groups with carbon number one to eight (C₁₋₈), substituent alkoxy groups with carbon number one to eight (C₁₋₈), non-substituent alkoxy groups with carbon number one to eight (C₁₋₈), alkylate groups with carbon number one to eight (C₁₋₈), nitrogen heterocyclic group, carboxyl group, nitro group, adamantyl carbonyl group, adamantyl group, alkenyl group, alkynyl group, amino group, azo group, aryl group, aryloxy group, arylcarbonyl group, aryloxycarbonyl group, arylcarbonyloxy group, aryloxycarbonyloxy group, alkylcarbonyl group, alkylcarbonyloxy group, alkoxycarbonyloxy group, alkoxycarbonyl group, carbamoyl group, cyanate group, cyano group, formyl group, formyloxy group, heterocyclic group, isothiocyanate group, isocyano group, isocyanate group, nitroso group, perfluoroalkyl group, perfluoroalkoxy group, sulfinyl group, sulfonyl group, silyl group, thiocyanate group, wherein R¹¹ and R¹² represent same or different chemical groups, and may be one selected from a group comprising hydrogen atom, nitro group, substituent or non-substituent alkyl groups with carbon number one to eight (C₁₋₈). A second substrate is covered on the recording damascene layers as a cover layer.

Further, the present invention provides a structure of a fluorescent storage media, comprising at least a first substrate, a recording stacked multilayer structure and a second substrate. The first substrate is a transparent substrate having a signal surface. The recording stacked multilayer structure is formed on the signal surface, and the recording stacked multilayer structure comprises more than one layer of fluorescent thin film, and in case of a recording stacked multilayer structure, there is an isolation layer disposed in between the two adjacent fluorescent thin films. The material of the fluorescent thin film comprises the fluorescent dye of the present invention having the chemical structure (I) as described above. The material of the isolation layer may comprise a dielectric layer or a polymer layer. The material of the dielectric layer comprises but not limited to, zinc sulfide-silicon dioxide (“ZnS—SiO2”), zinc sulfide (“ZnS”), aluminum nitride (“AlN”), silicon nitride (“SiN”) or Silica aerogel. The second substrate is formed over the recording stacked multilayer structure as a cover layer.

Furthermore, the present invention provides a manufacturing method of a fluorescent storage media, in which the method comprises providing a first transparent substrate having a signal surface, preparing a transparent polymer solution by dissolving a polymer material in an organic solvent, dissolving a fluorescent dye in the transparent polymer solution to obtain a dye solution, wherein the fluorescent dye comprises a chemical structure (I), coating the dye solution on the first transparent substrate, baking the resulting structure to form a fluorescent thin film, and forming a second substrate over the fluorescent thin films as a cover layer.

According to another aspect of the present invention, more than one fluorescent thin film may be formed by repeating the steps of forming the fluorescent thin film according to the method described above followed by forming an isolation layer in between two consecutive adjacent fluorescent films before the step of forming the second substrate. Thus, a multilayer storage media structure is formed.

According to another aspect of the present invention, before the step of forming the second substrate, a reflective layer is plated on the second substrate, to enhance the strength of the fluorescent storage media in order to prolong the life of the disc.

The fluorescent dye of the present invention is highly sensitive to short wavelength laser having a wavelength less than 500 nm, therefore, the fluorescent storage media using the fluorescent dye of the present invention is capable of using a short wavelength laser having a wavelength less than 500 nm as an excitation source. When using a short wavelength laser to excite the recording layer of the fluorescent storage media of the present invention, a crocus fluorescence is spontaneously induced and the induced fluorescence has a fluorescence radiation wavelength larger than 500 nm, and a reading operation signal of a fluorescent storage media is provided through detecting the strength of the fluorescence radiation.

Furthermore, because the fluorescent dye of the present invention has a sizable Stoke's shift, and therefore the fluorescent thin film with a sizable Stoke's shift may separate the wavelength of an incident laser beam from the induced fluorescence easily by filters. Therefore the cross-talk between the incident laser beam and the fluorescence radiation can be effectively avoided, and only the intensity of the fluorescence radiation can be exactly detected and provided for the reading operation signal of the information. As the absorption of the fluorescence radiation by the dye is low, therefore the decay of the fluorescence radiation can be avoided.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is a ultraviolet (“UV”)/visible spectrum of a film of the fluorescent dye (1) of the present invention formed on a polycarbonate substrate;

FIG. 2 is a fluorescence spectrum of a film of the fluorescent dye (1) of the present invention formed on a polycarbonate substrate;

FIG. 3 is a photographic view of a yellow spot fluorescence signal induced by exciting the read-only fluorescent disc manufactured using the fluorescent dye of the present invention by a wavelength 405 nm blue laser;

FIG. 4 is a distribution graph of the fluorescence intensities of the white line regions shown in FIG. 3;

FIG. 5 is a photographic view of the yellow bright belt of the fluorescence signal induced by exciting the write-once only fluorescent disc manufactured using the fluorescent dye of the present invention by a wavelength 405 nm blue laser in a copy operation;

FIG. 6 is a sectional view illustrating the structure of the fluorescent storage media of the present invention; and

FIG. 7 illustrates a process flow chart of the manufacturing method of the fluorescent storage media of the present invention.

### DESCRIPTION OF THE PREFERRED EMBODIMENTS

The present invention discloses a fluorescent dye, a structure of a fluorescent storage media and manufacturing method using thereof. The fluorescent dye of the present invention provides a short wavelength laser having a wavelength less than 500 nm for reading and saving operations. The fluorescent dye of the present invention includes di-phenylethene compounds having a following chemical structure (I):

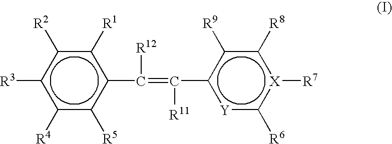

wherein X may be a carbon atom or a nitrogen atom. Y may be a carbon atom attaching a side chain (i.e. C—R⁹) or an oxygen atom. R¹, R², R³, R⁴, R⁵, R⁶, R⁷, R⁸ and R⁹ represent same or different chemical groups, and may be one selected from a group comprising hydrogen atom, halogen atom, substituent alkyl groups with carbon number one to eight (C₁₋₈), non-substituent alkyl groups with carbon number one to eight (C₁₋₈), substituent alkoxy groups with carbon number one to eight (C₁₋₈), non-substituent alkoxy groups with carbon number one to eight (C₁₋₈), alkylate groups with carbon number one to eight (C₁₋₈), nitrogen heterocyclic group, carboxyl group, nitro group, adamantyl carbonyl group, adamantyl group, alkenyl group, alkynyl group, amino group, azo group, aryl group, aryloxy group, arylcarbonyl group, aryloxycarbonyl group, arylcarbonyloxy group, aryloxycarbonyloxy group, alkylcarbonyl group, alkylcarbonyloxy group, alkoxycarbonyloxy group, alkoxycarbonyl group, carbamoyl group, cyanate group, cyano group, formyl group, formyloxy group, heterocyclic group, isothiocyanate group, isocyano group, isocyanate group, nitroso group, perfluoroalkyl group, perfluoroalkoxy group, sulfinyl group, sulfonyl group, silyl group, thiocyanate group, wherein R¹¹ and R¹² represent same or different chemical groups, and may be one selected from a group comprising hydrogen atom, nitro group, substituent or non-substituent alkyl groups with carbon number one to eight (C₁₋₈).

For the purpose of illustrating the excited radiation property of the fluorescent dye of the present invention, the chemical names and chemical structures of some of the fluorescent dyes of the present invention are shown in the following examples 1 to 6 will be described as follows. But however, the claims of the present invention are not limited to the experimental examples 1 to 6.

### EXAMPLE 1

The chemical structure of N,N-Dimethyl-N-{4-[(E)-2-(4-ntirophenyl)-1-ethenyl]phenyl}-amine (“Stil-1”) is shown below:

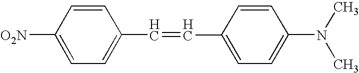

### EXAMPLE 2

The chemical structure of 1-(4-{(E)-2-[4-(dibutylamino)phenyl]-1-ethenyl}-phenyl)-2,2,2-trifluoro-1-ethanone (“Stil-2”) is shown below:

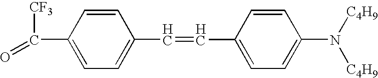

### EXAMPLE 3

The chemical structure of 4-[trans-4-(Diethylamino)styryl]-1-methyl-pyridinium 7,7,8,8-tetracyano-cyanoquinodimethane (“Stil-3”) is shown below:

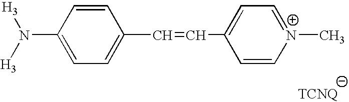

### EXAMPLE 4

The chemical structure of [2-[2-[4-(Dimethylamino)-phenyl]ethenyl]-6-methyl-4H-pyran-4-ylidene]propanedinitrile] (“DCM-1”) is shown below:

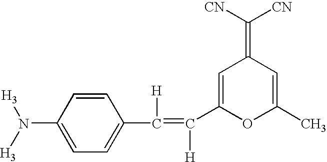

### EXAMPLE 5

The chemical structure of [2-[2-[4-(Diphenylamino)-phenyl]ethenyl]-6-methyl-4H-pyran-4-ylidene]propanedinitrile] (“DCM-2”) is shown below:

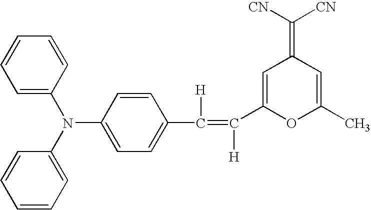

### EXAMPLE 6

The chemical structure of DCM-3 is shown below:

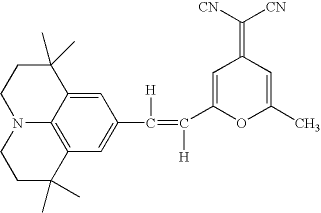

The fluorescent dyes described above is highly soluble in propylene glycol monomethyl ether (“PM”), and the fluorescence efficiency, the maximum absorbance of the incident excitation source and the fluorescence radiation wavelength are measured. The results are listed in table 1 below:

TABLE 1

| | | Fluorescence | |
|---|---|---|---|
| | UV Maximum | radiation | Fluorescence |
| Compound | absorption λₘₐₓ (nm) | λₘₐₓ (nm) | efficiency |
| (1) Stil-1 | 432 nm | 555 nm | 0.79 |
| (2) Stil-2 | 446 nm | 596 nm | 0.64 |
| (2) Stil-3 | 480 nm | 595 nm | 0.52 |
| (4) DCM-1 | 468 nm | 604 nm | 0.79 |
| (5) DCM-2 | 503 nm | 622 nm | 0.54 |
| (6) DCM-3 | 510 nm | 616 nm | 0.78 |

The results of Table 1 infer that the fluorescent storage media using the fluorescent dye of the present invention is suitable for making a recording media for saving and reading operations using a short wavelength laser with a wavelength less than 500 nm exhibiting a maximum absorbance of the incident excitation source and induce fluorescence radiation, and more particularly by using the short wavelength blue laser (with a wavelength of 405 nm). The fluorescent dye of the present invention has an excellent fluorescence quantum efficiency, wherein, the efficiency of all the fluorescent dyes of the present invention is larger than 50%.

The fluorescent dye (1), for example, Stil-1, is used for forming a recording stacked multilayer structure. First of all, the Stil-1 is dissolved in a polymer solution to prepare a dye solution, preferably a mole concentration 10⁻³ M is prepared, wherein the polymer solution is propylene glycol monomethyl ether (“PM”) including a 5 wt % polyvinyl butyral (“PVB”). Next, the dye solution is coated on a transparent substrate. The material of the substrate may be comprised of polycarbonate. and the resulting structure is subjected to a baking process to form a fluorescent thin film. Thus, a recording layer is formed. Subsequently, the optical properties of the fluorescent thin film are measured.

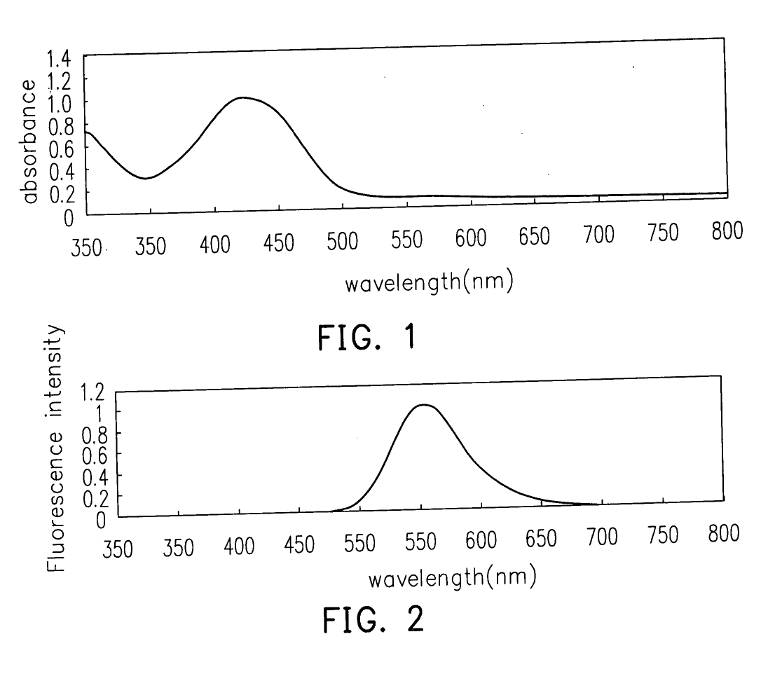

As shown in FIG. 1, the fluorescent thin film of the present invention exhibits a maximum absorption at a wavelength of about 441 nm. Further, as shown in FIG. 2, the fluorescent thin film exhibits a maximum fluorescence radiation at a wavelength of about 556 nm. (λ em=556 nm)

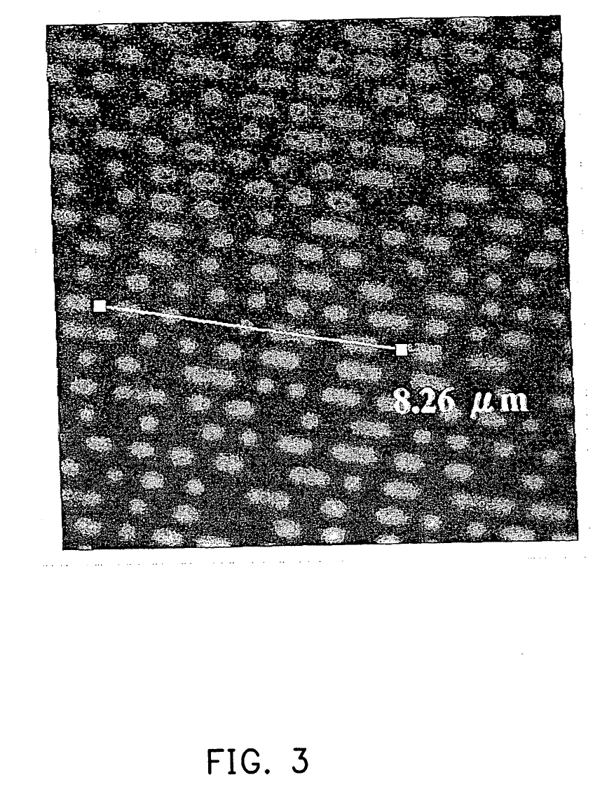

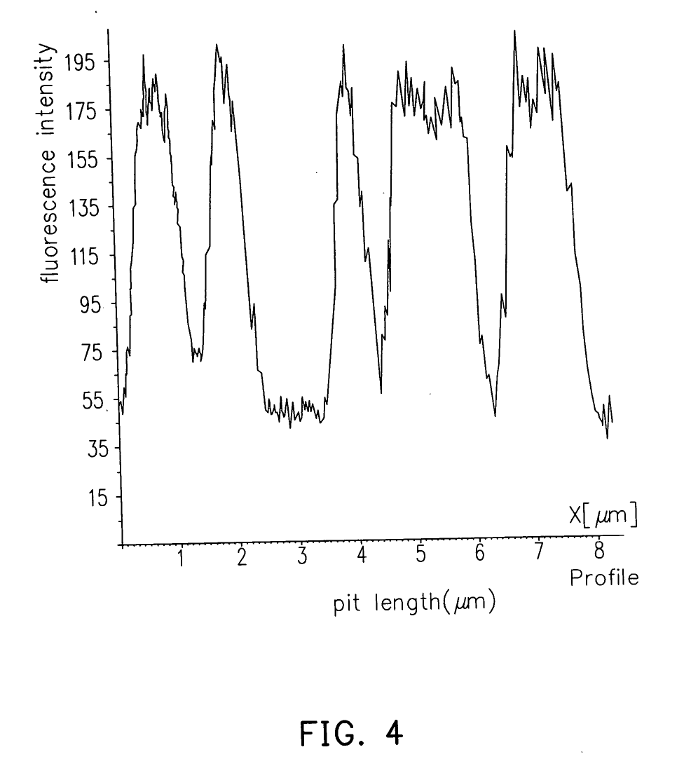

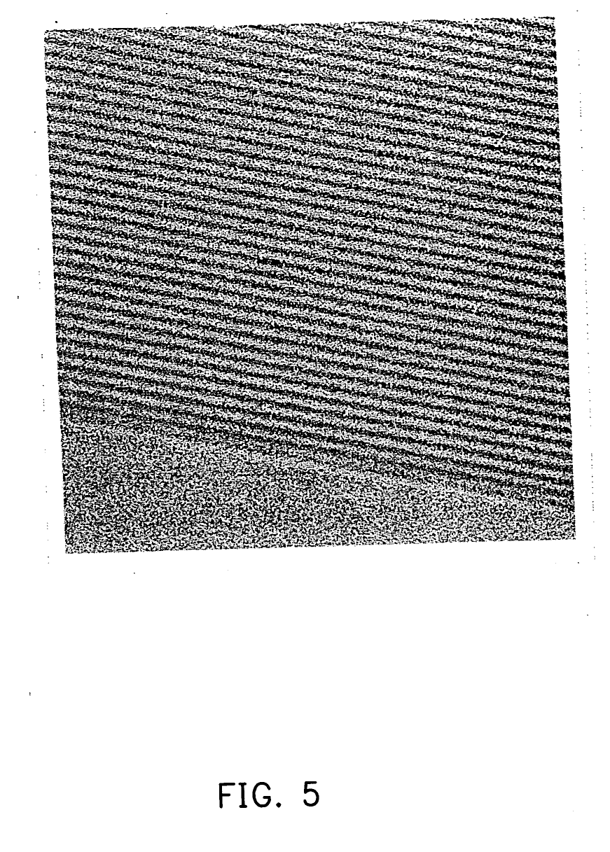

Moreover, the fluorescent thin film of the present invention formed on a read only digital versatile disc substrate (DVD-ROM substrate), also excited by a 405 nm wavelength blue laser was photographed, and is shown in FIG. 3, showing yellow light spots of light and shade spots. The light and shade spots are due to the difference in the intensity of the fluorescence. Accordingly the encoding of the information 0 and 1 principle can be correlated by the light and shade spots. Furthermore, FIG. 4 shows a graph of distribution of the intensity of the fluorescence of the white line regions in FIG. 3. Comparing the diagram in FIG. 4 to that in FIG. 3, the bright spot region correspond to an intense fluorescence, and the dark spot region correspond to a weak fluorescence. The relative intensity ratio of the fluorescence is about 71%. The relative intensity ratio of the fluorescence can be expressed as follows:

Relative intensity ratio=(the strength of the bright spot—the strength of the dark spot)/(the strength of the bright spot).

Moreover, as shown in FIG. 1, by copying one layer of a write-once digital versatile disc substrate (DVD-ROM substrate) once on the first fluorescent substrate, and coating the dye solution on the copying substrate to form a layer, then after exciting by a 405 nm wavelength blue laser, a diagram of yellow light belts with light and shade is obtained.

Thus, the fluorescent storage media comprising a recording stacked multilayer structure comprising the fluorescent dye of the present invention, is highly sensitive to a short wavelength laser having a wavelength less than 500 nm, and therefore can be used as an excitation source of the fluorescent storage media. When using a short wavelength laser to excite the recording layer of the fluorescent storage media of the present invention, the crocus fluorescence induced spontaneously has a fluorescence radiation wavelength larger than 500 nm. A reading signal can be provided by detecting the strength of the fluorescence radiation.

Furthermore, because the fluorescent dye of the present invention has a sizable Stoke's shift. Stoke's shift means, as the fluorescent matter returns to the ground state from the excited state, it releases photons with a energy (i.e., a fluorescence), in doing so, some energy is lost in a solution or in a solid state, and therefore the emitted fluorescence generally has a lower energy and a longer wavelength compared to that of the incident excitation source. That is, the difference between the incident excitation source and the emitted fluorescence is called a Stoke's shift). Accordingly the fluorescent thin film of the present invention with a sizable Stoke's shift makes it possible to separate the wavelength of a incident laser beam from the induced fluorescence radiation easily by using filters. Therefore cross-talk between the incident laser beam and the fluorescence radiation may be avoided, and thus the intensity of the fluorescence radiation can be precisely detected as reading signal of the information. The intensity of the fluorescence radiation absorbed by the dye can be decreased, and the decay of the intensity of the fluorescence radiation is avoided.

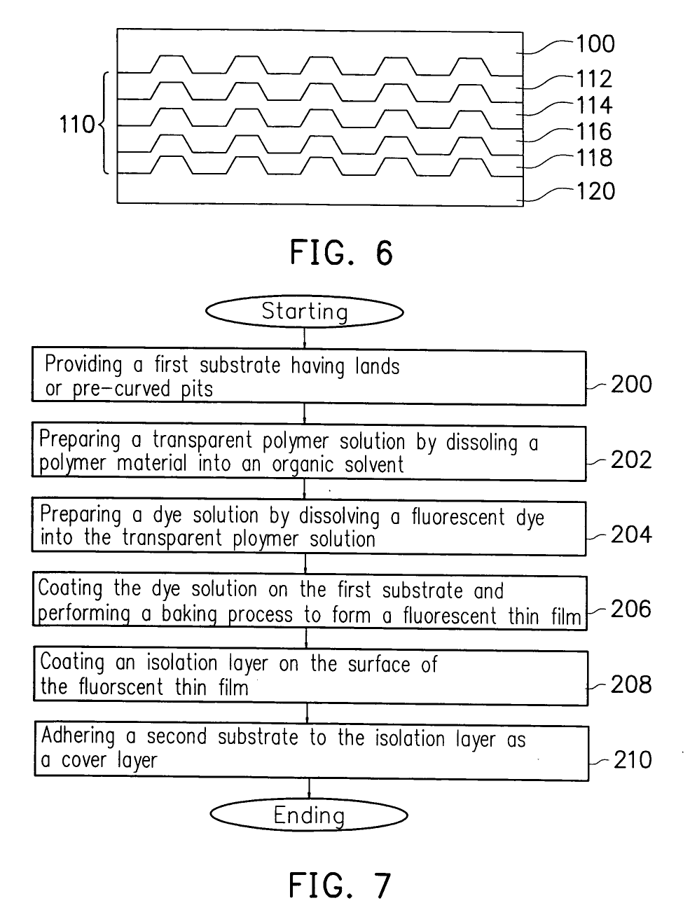

The structure of the fluorescent storage media of the present invention is described as follows. Referring to FIG. 6, the fluorescent storage media of the present invention comprises at least a first substrate 100, a recording stacked multilayer structure 110 disposed over the first substrate, and a second substrate disposed over the recording stacked multilayer structure. The first substrate 100, for example, is a transparent substrate having lands or pre-curved pits. The recording stacked multilayer structure 110 comprises at least a first fluorescent thin film 112, a first isolation layer 114 disposed over the first fluorescent thin film 112, a second fluorescent thin film 116 disposed over the first isolation layer 114, and a second isolation layer 118 disposed over the second fluorescent thin film 116. The first fluorescence thin film 112 and the second fluorescence thin film 116 are composed of an organic violet fluorescent dye compound, wherein the organic violet fluorescent dye compound, for example, comprises the fluorescent dye of the present invention having the chemical structure (I). A thickness of a fluorescence thin film, for example, is about 50 nm to 1000 nm.

The lands or pre-curved pits in the transparent substrate are used to provide a signal surface for the laser tracking of the pick-up head of the laser. The recording stacked multilayer structure 110 described above is illustrated by an example of comprising two fluorescent thin films 112, 116 and two isolation layers 114, 118. However, it is to be understood that the recording stacked multilayer structure may be comprises more than two layers of fluorescent thin films with an isolation layer isolating two consecutive fluorescent thin films.

Referring to FIG. 7, a process flow chart describes the steps of the manufacturing method of the fluorescent storage media of the present invention. The manufacturing method comprises, providing a first substrate 100 (step 200) having lands or pre-curved pits. The lands or pre-curved pits in the first substrate are used to provide a signal surface for the laser tracking of the pick-up head of the laser. The material of the first substrate is comprised of, but not limited to, polyster, polycarbonate (PC), polymethylmethacrylate (PMMA), metallocene catalyzed cyclic olefin copolymers (mCOC).

Next, a polymer material is dissolved in an organic solvent and a transparent polymer solution is prepared (step 202). The polymer material includes, but not limited to, chitin, cellulose or polyvinyl butyral. The organic solvent comprises alcohol with carbon number one to six, ketone with carbon number one to six, ether with carbon number one to six, dibutyl ether (“DBE”), halogen compounds, amide or methylcyclohexane (“MCH”). For example, the alcohol with carbon number one to six comprises methanol, ethanol, isopropanol, diacetonalchol (“DAA”), 2,2,3,3-tetrafluoropropanol, trichloroethanol, 2-chloroethanol, octafluoropentanol, hexafluorobutanol, propylene glycol monoethyl ether or propylene glycol monoethyl acetate. The ketone with carbon number one to six comprise acetone, methyl isobutyl ketone, (“MIBK”), methyl ethyl ketone, (“MEK”), or 3-hydroxy-3-methyl-2-butanone. The halogen compounds comprise chloroform, dichloromethane or 1-chlorobutane. The amide comprises, such as dimethylformamide (“DHF”) or dimethylacetamide (“DMA”). The concentration of the transparent polymer solution is about 1 wt % to about 20 wt %, and a preferably concentration is about 1 wt % to about 5 wt %.

Next, a fluorescent dye, for example, a di-phenylethene compound, of the present invention is dissolved in the above transparent polymer solution, and a dye solution is obtained (step 204). A dye solution of a molar concentration about 10⁻⁷ M to 10⁻² M is prepared.

Next, the dye solution is coated on the first substrate and then the resulting structure is subjected to a baking process to form a fluorescent thin film on the first substrate (step 206). The method of coating the dye solution on the first substrate comprises, but not limited to, a spin coating method, a roll-pressing coating method, a dip coating method or an inkjet printing method.

Next, an isolation layer is coated on the fluorescent thin film (step 208). The isolation layer includes, such as a dielectric layer of a thickness of about 50 nm to about 200 nm, or a polymer layer of a thickness about 1 um to about 20 um. The material of the dielectric layer comprises zinc sulfide-silicon dioxide (“ZnS—SiO2”), zinc sulfide (“ZnS”), aluminum nitride (“AlN”), silicon nitride (“SiN”) or Silica aerogel. Next, a second substrate is adhered to the isolation layer as a cover layer (step 210). Likewise, the material of the second substrate includes, for example polyster, polycarbonate (PC), polymethylmethacrylate (PMMA), metallocene catalyzed cyclic olefin copolymers (mCOC). The method of adhering the other substrate to the isolation layer as a cover layer includes, but not limited to, a spin coating, a screen printing, a hot melt glue coating or a double sided tape adhesion.

The manufacturing steps 206 to the step 208 may be repeated more than once before the step 210, if a recording stacked multilayer structure comprising a plurality of fluorescent thin films is desired. To enhance the strength of the measured fluorescence and the conservation life of the disc, it is preferable to form a reflective layer between the second substrate and the recording stacked multilayer structure with a thickness of about 50 nm to 300 nm. However, it is to be understood that in case the fluorescent storage media comprises a single fluorescent thin film, then the reflective layer can be disposed between the fluorescent thin film and the second substrate. The material of the reflective layer includes, but not limited to, gold, silver, aluminum, silicon, copper, alloy of silver and titanium.

The fluorescent storage media manufactured using the fluorescent dye of the present invention is suitable for the saving and reading operations using a short wavelength laser with a wavelength less than 500 nm. Because the fluorescent dye of the manufacturing of a fluorescent storage media of the present invention has a sizable Stoke's shift, therefore the cross-talk between the incident laser beam and the fluorescence radiation can be effectively avoided. The intensity of the fluorescence radiation can be precisely detected and provided as reading signal of the information. As the intensity of the fluorescence radiation absorbed by the dye is low, therefore the decay of the intensity of the fluorescence radiation can be effectively avoided.

Furthermore, when using a short wavelength laser to excite the fluorescent thin film (recording layer) of the fluorescent storage media of the present invention, a crocus fluorescence is spontaneously induced, and the induced fluorescence has a fluorescence radiation wavelength larger than 500 nm. And a reading signal of a fluorescent storage media is provided through detecting the intensity of the fluorescence radiation.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. A fluorescent storage media, comprising:
at least a recording layer comprising fluorescent dye having a following chemical structure (I): 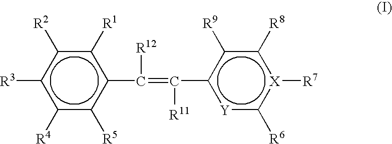
wherein X comprises a carbon atom, Y comprises a carbon atom attaching side chain (i.e. C—R⁹) or an oxygen atom, and R¹, R², R³, R⁴, R⁵, R⁶, R⁷, R⁸ and R⁹ represent same or different chemical groups, and can be one selected from a group consisting hydrogen atom, halogen atom, substituent alkyl groups with carbon number one to eight (C₁₋₈), non-substituent alkyl groups with carbon number one to eight (C₁₋₈), substituent alkoxy groups with carbon number one to eight (C₁₋₈), non-substituent alkoxy groups with carbon number one to eight (C₁₋₈), alkylate groups with carbon number one to eight (C₁₋₈), nitrogen heterocyclic group, carboxyl group, nitro group, adamantyl carbonyl group, adamantyl group, alkenyl group, alkynyl group, amino group, azo group, aryl group, aryloxy group, arylcarbonyl group, aryloxycarbonyl group, arylcarbonyloxy group, aryloxycarbonyloxy group, alkylcarbonyl group, alkylcarbonyloxy group, alkoxycarbonyloxy group, alkoxycarbonyl group, carbamoyl group, cyanate group), cyano group, formyl group, formyloxy group, heterocyclic group, isothiocyanate group, isocyano group, isocyanate group, nitroso group, perfluoroalkyl group, perfluoroalkoxy group, sulfinyl group, sulfonyl group, silyl group, thiocyanate group, and wherein R¹¹ and R¹² represent same or different chemical groups, and can be one selected from a group consisting hydrogen atom, nitro group, substituent or non-substituent alkyl groups with carbon number one to eight (C₁₋₈).

2. A fluorescent storage media, comprising:
a first transparent substrate having a signal surface;
a recording stacked multilayer structure, formed on the signal surface of the first transparent substrate, wherein the recording stacked multilayer structure comprises a plurality of fluorescent thin films, wherein an isolation layer is disposed between the two consecutive fluorescent thin films, and wherein a material of the fluorescent thin films comprises a compound having a following chemical structure (I) 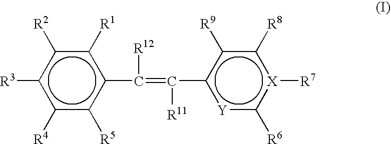
wherein X comprises a carbon atom, Y comprises a carbon atom attaching side chain (i.e. C—R⁹) or an oxygen atom and R¹, R², R³, R⁴, R⁵, R⁶, R⁷, R⁸ and R⁹ represent same or different chemical groups, and can be one selected from a group consisting hydrogen atom, halogen atom, substituent alkyl groups with carbon number one to eight (C₁₋₈), non-substituent alkyl groups with carbon number one to eight (C₁₋₈), substituent alkoxy groups with carbon number one to eight (C₁₋₈), non-substituent alkoxy groups with carbon number one to eight (C₁₋₈), alkylate groups with carbon number one to eight (C₁₋₈), nitrogen heterocyclic group, carboxyl group, nitro group, adamantyl carbonyl group, adamantyl group, alkenyl group, alkynyl group, amino group, azo group, aryl group, aryloxy group, arylcarbonyl group, aryloxycarbonyl group, arylcarbonyloxy group, aryloxycarbonyloxy group, alkylcarbonyl group, alkylcarbonyloxy group, alkoxycarbonyloxy group, alkoxycarbonyl group, carbamoyl group, cyanate group), cyano group, formyl group, formyloxy group, heterocyclic group, isothiocyanate group, isocyano group, isocyanate group, nitroso group, perfluoroalkyl group, perfluoroalkoxy group, sulfinyl group, sulfonyl group, silyl group, thiocyanate group, wherein R¹¹ and R¹² represent same or different chemical groups, and can be one selected from a group consisting hydrogen atom, nitro group, substituent or non-substituent alkyl groups with carbon number one to eight (C₁₋₈); and
a second substrate, formed over the recording stacked multilayer structure.

3. The fluorescent storage media of claim 2, wherein the second substrate comprises a transparent substrate.

4. The fluorescent storage media of claim 2, wherein a material of the first transparent substrate and the second substrate comprise polyster, polycarbonate (PC), polymethylmethacrylate (PMMA), or metallocene catalyzed cyclic olefin copolymers (mCOC).

5. The fluorescent storage media of claim 2, wherein a thickness of the fluorescent thin film is in a range of about 50 nm to 1000 nm.

6. The fluorescent storage media of claim 2, wherein the isolation layer is one selected from a group consisting of a dielectric layer and a polymer layer.

7. The fluorescent storage media of claim 6, wherein a thickness of the dielectric layer is in a range of about 50 nm to 200 nm.

8. The fluorescent storage media of claim 6, wherein the material of the dielectric layer comprises zinc sulfide-silicon dioxide (“ZnS—SiO2”), zinc sulfide (“ZnS”), aluminum nitride (“AlN”), silicon nitride (“SiN”) or Silica aerogel.

9. The fluorescent storage media of claim 6, wherein a thickness of the polymer layer is in a range of about 1 um to 20 um.

10. The fluorescent storage media of claim 2, wherein the fluorescent storage media further comprises a reflective layer disposed between the second substrate and the recording stacked multilayer structure.

11. The fluorescent storage media of claim 2, wherein a material of the reflective layer comprises gold, silver, aluminum, silicon, copper, alloy of silver and titanium, alloy of silver and chromium or alloy of silver and copper.

12. The fluorescent storage media of claim 2, wherein the thickness of the reflective layer is in a range of about 50 nm to 300 nm.

13-35. (canceled)
